(19) United States
(12) Patent Application Publication
(10) Pub. No.: US 2016/0303043 A1
(43) Pub. Date: Oct. 20, 2016

(54) SELF-FOAMING COMPOSITIONS AND METHODS

(71) Applicant: Kate Somerville Skincare, LLC, Beverly Hills, CA (US)

(72) Inventor: Fred Khoury, Chatsworth, CA (US)

(73) Assignee: Kate Somerville Skincare, LLC, Beverly Hills, CA (US)

(21) Appl. No.: 14/790,900

(22) Filed: Jul. 2, 2015

Related U.S. Application Data
(60) Provisional application No. 62/186,643, filed on Jun. 30, 2015.
(60) Provisional application No. 62/148,393, filed on Apr. 16, 2015.

(51) Int. Cl. A61K 9/12; A61K 47/14; A61K 47/20; B65D 85/00; A61K 47/10; A61K 47/12; A61K 47/08; A61K 33/04; A61K 47/26

(52) U.S. Cl. CPC A61K 9/122; A61K 33/04; A61K 47/14; A61K 47/20; A61K 47/26; A61K 47/10; A61K 47/12; A61K 47/08; B65D 85/70

(57) ABSTRACT

A self-foaming composition can include at least one perfluoro compound, at least one surfactant, and at least one fatty component. The composition, when released from a sealed vessel, forms a foam without the need to add any other components. The composition can include an active agent, e.g., for skin care, such as an anti-acne agent.

### CLAIM OF PRIORITY

This application claims priority to provisional U.S. Patent Application No. 62/148,393, filed Apr. 16, 2015, and to provisional U.S. Patent Application No. 62/186,643, filed Jun. 30, 2015, each of which is incorporated by reference in its entirety.

### TECHNICAL FIELD

This disclosure relates to self-foaming compositions and methods of making and using them.

### BACKGROUND

Compositions for treating acne frequently take the form of a spot treatment, such as a cream or lotion that is applied only at the location of a blemish. Applying the treatment to the entire face can help prevent the formation of blemishes in areas that are clear. Currently available spot treatments may be too harsh to the skin to be suitable for use on the entire face. There is a need for acne treatments suitable for broader use than a spot treatment.

### SUMMARY

In one aspect, a self-foaming composition includes at least one perfluoro compound; at least one surfactant; and at least one fatty component selected from a fatty alcohol and/or a fatty acid; where the composition, when released from a sealed vessel, forms a foam.

In another aspect, a method of making a self-foaming composition includes forming a composition by combining at least one perfluoro compound; at least one surfactant; and at least one fatty component selected from a fatty alcohol and/or a fatty acid; and enclosing the composition in a sealed vessel; where the composition, when released from the sealed vessel, forms a foam.

The composition can include from about 0.1% w/w to about 25% w/w of surfactant(s). The surfactant(s) can be selected from glycereth-18 ethylhexanoate, glycereth-18, sodium C14-16 olefin sulfonate, sodium cocoamphoacetate, cocamidopropyl hydroxysultaine, coco glucoside, and mixtures thereof.

The composition can include from about 0.1% w/w to about 10% w/w of the fatty component. The fatty component can be selected from cetyl alcohol, coconut alcohol, oleyl alcohol, stearyl alcohol, arachidyl alcohol, batyl alcohol, behenyl alcohol, lauric acid, myristic acid, palmitic acid, stearic acid, arachidic acid, behenic acid, myristoleic acid, palmitoleic acid, oleic acid, linoleic acid, linolenic acid, arachadonic acid, and combinations thereof. The composition can include at least two fatty components.

The composition of can further include a skin care active agent. The active agent can be an anti-acne agent. The anti-acne agent can be selected from sulfur, colloidal sulfur, benzoyl peroxide, and salicylic acid, or a combination thereof. The perfluoro compound can be a perfluoro ether.

In some embodiments, the composition includes water (40-60% w/w); at least one of: glycereth-18 ethylhexanoate (0-2.5% w/w), glycereth-18 (0-2.5% w/w), sodium C14-16 olefin sulfonate (5-15% w/w), sodium cocoamphoacetate (1-5% w/w), cocamidopropyl hydroxysultaine (0.1-1.5% w/w), and coco glucoside (2-8% w/w); and at least one of: coconut alcohol (2-8% % w/w), behenyl alcohol (0.1-2.5% w/w), and stearyl alcohol (0.1-5% w/w).

In some embodiments, the composition includes Water (Aqua), Xanthan Gum, Acrylates Copolymer, Glycereth-18 Ethylhexanoate, Glycereth-18, Sodium C14-16 Olefin Sulfonate, Sodium Cocoamphoacetate, Cocamidopropyl Hydroxysultaine, Propanediol, Phenoxyethanol, Ethylhexylglycerin, Coco Glucoside, Coconut Alcohol, Behenyl Alcohol, Stearyl Alcohol, Ethylhexylglycerin, Phenoxyethanol, and colloidal sulfur.

The composition can be in a sealed bag-on-valve container.

Additional features and aspects will be apparent from the description and claims.

### DETAILED DESCRIPTION

A composition including at least one perfluoro compound, at least one surfactant, and at least one fatty alcohol can provide a self-foaming formulation for cosmetics. The composition may be conveniently stored as a liquid in a sealed airless container. When the composition is dispensed from the container, it forms a foam. The foam can be formed while one or more components, for example, one or more perfluoro compound, evaporate from the composition.

A “self-foaming composition” refers to a composition that forms a foam when released or dispensed from a sealed container, without the need for mixing or addition of other components, e.g., air or another gas. The self-foaming action can be facilitated by evaporation of one or more components of the composition. Storage in a sealed container prevents the premature evaporation of such components, so that the foam is formed only when desired, i.e., when released or dispensed from the sealed container.

The self-foaming composition can be an aqueous composition (such as a solution, oil-in-water emulsion, water-in-oil emulsion, or aqueous mixture) including one or more perfluoro compounds, one or more surfactants, and one or more fatty alcohols. The self-foaming properties of the composition are influenced by the nature and concentration of these components.

Perfluoro Compounds

A “perfluoro compound” as used herein refers to a volatile organic compound having at least one perfluoroalkyl or perfluoroalkenyl group. Perfluoro compounds include, for example, perfluorocarbons and perfluoroethers. A perfluoroether can have the formula R—O—R′ where at least one of R and R′ is perfluoroalkyl or perfluoroalkenyl. The other of R and R′ can be perfluoroalkyl, perfluoroalkenyl, alkyl, alkenyl, or another functional group. In some cases, R can be perfluoroalkyl and R′ can be alkyl.

Some examples of perfluoro compounds include methyl perfluorobutyl ether, methyl perfluoroisobutyl ether, ethyl perfluorobutyl ether, ethyl perfluoroisobutyl ether, perfluoropropane, perfluorobutane, perfluoropentane, perfluorohexane, perfluorodecalin, perfluorodimethyl-cyclohexane, perfluoroperhydrophenanthrene, pentafluoro-propane, perfluorotripropylamine, C₆-C₉ perfluoroalkanes, perfluoroperhydrofluoranthrene, perfluorodecalin, perfluoroperhydro-phenanthrene, bis(perfluor-hexyl)-1,2-ethene, perfluoro-1,3-dimethylcyclohexane, perfluoro-methyldecalin, perfluoroisopropyldecalin, a mixture of perfluorodixylylmethane and perfluorodixylylethane, and/or a mixture of perfluoroperhydrophenanthrene and perfluoro n-butyl decalin.

In some embodiments, the concentration of the perfluoro compounds can be up to, about, or not more than 1%, 5%, 6%, 7%, 8%, 9%, 10%, 11%, 12%, 13%, 14%, 15%, 16%, 18%, 20%, or 25% by weight.

Surfactants

A number of suitable surfactants are known in the art, for example, those described in the International Cosmetic Ingredient Dictionary and Handbook. As used herein, “surfactants” refers without limitation to cationic, anionic, amphoteric, ethoxylated, non-ethoxylated, and amino acid surfactants.

Suitable anionic surfactants include but are not limited to alkyl sulfates, alkyl ether sulfates, alkaryl sulfonates, alkyl succinates, alkyl sulfosuccinates, N-alkoyl sarcosinates, alkyl phosphates, alkyl ether phosphates, alkyl ether carboxylates, alkylamino acids, alkyl peptides, alkoyl taurates, carboxylic acids, acyl and alkyl glutamates, alkyl isethionates, and alpha-olefin sulfonates, especially their sodium, potassium, magnesium, ammonium and mono-, di- and triethanolamine salts. The alkyl groups generally contain from 8 to 18 carbon atoms and may be unsaturated. The alkyl ether sulfates, alkyl ether phosphates and alkyl ether carboxylates may contain from 1 to 10 ethylene oxide or propylene oxide units per molecule, and preferably contain 1 to 3 ethylene oxide units per molecule.

Examples of suitable anionic surfactants include sodium and ammonium lauryl ether sulfate (with 1, 2, and 3 moles of ethylene oxide), sodium, ammonium, and triethanolamine lauryl sulfate, disodium laureth sulfosuccinate, sodium cocoyl isethionate, sodium C12-14 olefin sulfonate, sodium C14-16 olefin sulfonate, sodium laureth-6 carboxylate, sodium C12-15 pareth sulfate, sodium methyl cocoyl taurate, sodium dodecylbenzene sulfonate, sodium cocoyl sarcosinate, triethanolamine monolauryl phosphate, and fatty acid soaps.

Preferred anionic surfactants include sodium C14-16 olefin sulfonate, disodium laureth sulfosuccinate, sodium methyl oleyl taurate, sodium lauroyl sarcosinate, sodium laureth-5 carboxylate, sodium lauryl ether sulfate, sodium lauryl sulfate, TEA lauryl sulfate, ammonium lauryl ether sulfate, ammonium lauryl sulfate, sodium lauroyl oat amino acids, disodium cocoyl glutamate, glyceryl stearate citrate, cocoyl sarcosine, sodium cocoyl isethionate, and mixtures thereof. Additional preferred anionic surfactants include sodium stearate or other salts of fatty acids, e.g., a sodium or potassium salt of lauric acid, myrisitic acid, palmitic acid, and stearic acid.

The nonionic surfactant can be any of the nonionic surfactants known or previously used in the art of aqueous surfactant compositions. Suitable nonionic surfactants include but are not limited to saturated or unsaturated (C6-C22) primary or secondary linear or branched chain acids, alcohols or phenols, alkyl ethoxylates, alkyl phenol alkoxylates, block alkylene oxide condensate of alkyl phenols, alkylene oxide condensates of alkanols, ethylene oxide/propylene oxide block copolymers, semi-polar nonionics (e.g., amine oxides and phosphine oxides), as well as alkyl amine oxides. Other suitable nonionics include mono- or di-alkyl alkanolamides and alkyl polysaccharides, sorbitan fatty acid esters, polyoxyethylene sorbitan fatty acid esters, polyoxyethylene sorbitol esters, polyoxyethylene acids, and polyoxyethylene alcohols. Examples of suitable nonionic surfactants include coco mono or diethanolamide, coco (di)glucoside, alkyl polyglucoside, cocamidopropyl and lauramine oxide, polysorbate 20, ethoxylated linear alcohols, coconut alcohol, lanolin alcohol, stearic acid, glyceryl stearate, PEG-100 stearate, and oleth 20.

Preferred nonionic surfactants include coco glucoside, decyl glucoside, lauryl glucoside, cocamide MEA, cocamide DEA, Triton X-100, nonoxynol-9, glycerol monolaurate, polysorbate 20, sorbitan monooleate, poloxamers, PEG-7 glycerol cocoate, PPG-5 ceteth-20, trideceth-12, stearamidopropyl dimethylamine, PEG-10 dimethicone, and mixtures thereof.

Amphoteric and zwitterionic surfactants are those compounds which have the capacity of behaving either as an acid or a base. Suitable materials include but are not limited to alkyl betaines, alkyl amidopropyl betaines, alkyl sulphobetaines, alkyl glycinates, alkyl carboxyglycinates, alkyl amphopropionates, alkyl amidopropyl hydroxysultaines, acyl taurates and acyl glutamates wherein the alkyl and acyl groups have from 8 to 18 carbon atoms. Examples include cocamidopropyl betaine, sodium cocoamphoacetate, cocamidopropyl hydroxysultaine, and sodium cocamphopropionate.

Preferred amphoteric and zwitterionic surfactants include sodium cocoamphoacetate, cocamidopropyl hydroxysultaine, lauryl hydroxysultaine, cocamidopropyl betaine, phospholipids, disodium cocoamphodiacetate, cocamphodiproprionate, and mixtures thereof.

Suitable cationic surfactants include but are not limited to alkyl amines, alkyl imidazolines, ethoxylated amines, quaternary compounds, and quaternized esters. In addition, alkyl amine oxides can behave as a cationic surfactant at a low pH. Examples include dicetyldimonium chloride, and cetrimonium chloride.

In some embodiments, the surfactant(s) in the self-foaming composition includes one or more of sodium C14-16 olefin sulfonate, sodium cocoamphoacetate, cocamidopropyl hydroxysultaine, coco glucoside, fatty alcohols, and mixtures thereof. In some embodiments, the composition can include 5-25% surfactants.

Fatty Components

A “fatty component,” as used herein, refers to a fatty alcohol or a fatty acid. Fatty alcohols include saturated or unsaturated C12-C22 alcohols, preferably C14-C22 alcohols, such as, for example, cetyl alcohol, coconut alcohol, oleyl alcohol, lauryl alcohol, stearyl alcohol, arachidyl alcohol, and behenyl alcohol, and the like. Fatty acids include saturated or unsaturated C12-C22 alcohols, preferably C14-C22 alcohols, such as, for example, lauric acid, myristic acid, palmitic acid, stearic acid, arachidic acid, behenic acid, myristoleic acid, palmitoleic acid, oleic acid, linoleic acid, linolenic acid, arachadonic acid, and the like. A number of suitable fatty alcohols and fatty acids are known in the art, for example, those described in the International Cosmetic Ingredient Dictionary and Handbook. In some cases, a fatty alcohol or a fatty acid can also be considered a surfactant. In some embodiments, the composition can include 5-25% w/w of fatty component(s).

Additional Components

Skilled workers will recognize other, optional ingredients that can be added to a formulation of the invention. In some cases, a component may be categorized in more than one of the following categories. Without limitation, some suitable optional components include:

Emulsifiers: including nonionic, cationic, anionic or polymeric emulsifiers; for example glyceryl stearate, cetearyl alcohol, cetearyl phosphate, behentrimonium chloride, polysorbate-20, acrylates copolymer, acrylaytes/C10-30 alkyl acrylate crosspolymer, and the like (0-20%).

Rheology modifiers: including, e.g., polyacrylic acid polymers, xanthan gum, cellulose gums, silicates, alginates, hydrocolloids, etc 0-1%).

Humectants: such as, e.g., propanediol, glycerin and other glycols, including butylene glycol (0-8%).

Emollients: such as, e.g, squalane, ethylhexyl palmitate, diisopropyl dimer dilinolcate, C12-15 Alkyl Benzoate, or Melalenca alternifolia oil (Tea Tree Leaf) (0-20%).

pH modifiers: such as, e.g., triethanolamine, sodium hydroxide, acidifiers, including citric acid (0-5%).

Antimicrobial agents: such as, e.g., salicylic acid, or preservatives, such as, e.g., phenoxyethanol, benzyl alcohol, glycereth-18 Ethylhexanoate, glycereth-18,or potassium sorbate (0-3%).

Aromas: in the form of fragrances or essential oils (0-5%).

Antioxidants: such as, e.g., ascorbic acid or derivatives thereof, including ascorbyl palmitate, ascorbyl glucoside, ascorbyl isostearate, sodium ascorbyl phosphate, magnesium ascorbyl phosphate, ethyl ascorbic acid, or aminopropyl ascorbyl phosphate; or tocopherol of derivatives thereof, including tocopheryl acetate, tocopheryl oleate, or tocopheryl linoleate.

Plant extracts: such as Avena Sativa (Oat) Extract; Oryza Sativa (Rice) Bran Extract; Boswellia Serrata Extract; Honey Extract; or others.

Colorants: For the use as a foundation for make-up, suitable colorants such as e.g., FD&C colors (colors which are certified and allowed by the US for the Food, Pharmaceutical, Cosmetics & Personal Care industry), iron oxides, titanium dioxides, including silicon-treated pigments, can be included, in the form of powder particle or pre-dispersions in various dispersants, such as castor oil, silanetriol, silicone, hydroxystearic Acid, mineral oils, or C₁₂-C₁₅ alkyl benzoate.

Preservatives.

Suitable buffers.

Actives

The self-foaming composition can include an active ingredient selected for a desired function, such as anti-wrinkle, moisturizing, anti-acne, or other cosmetic purpose. Some example of a suitable anti-acne active ingredients are sulfur, colloidal sulfur, benzoyl peroxide, and salicylic acid, or a combination thereof. In some embodiments, the anti-acne active ingredient can be colloidal sulfur.

In illustrative embodiments, the self-foaming composition can be formulated according to the Table 1. Table 1 is meant to be non-limiting, i.e., the self-foaming composition can include additional components as discussed above.

TABLE 1

| Component | Range (% w/w) | Range (% w/w) |
|---|---|---|
| Water | 30-80 | 40-60 |
| Surfactants | | |
| glycereth-18 ethylhexanoate | 0-5 | 0-2.5 |
| glycereth-18 | 0-5 | 0-2.5 |
| sodium C14-16 olefin sulfonate | 0-20 | 5-15 |
| sodium cocoamphoacetate | 0-10 | 1-5 |
| cocamidopropyl hydroxysultaine | 0-2 | 0.1-1.5 |
| coco glucoside | 0-10 | 2-8 |
| Fatty alcohols | | |
| coconut alcohol | 0-10 | 2-8 |
| behenyl alcohol | 0-5 | 0.1-2.5 |
| stearyl alcohol | 0-10 | 0.1-5 |
| Active agent | | |
| colloidal sulfur | 0-15 | 3-10 |
| perfluoro compound | | |
| ethyl perfluorobutyl ether | 0-10 | 0.1-5 |
| ethyl perfluoroisobutyl ether | | |

In illustrative embodiments, the self-foaming composition can be formulated according to the Table 2. Phases A-D are prepared and combined together. Phase E is added last and the composition placed in a sealed container.

Some of the blends in the table below are commercially available, for example, a blend of Glycereth-18 Ethylhexanoate and Glycereth-18 is available from Global Seven; and a blend of Water, Sodium C14-16 Olefin Sulfonate, Sodium Cocoamphoacetate, Cocamidopropyl Hydroxysultaine, Propanediol, Phenoxyethanol, Ethylhexylglycerin, and Sodium Chloride is available from Ross Organics. A blend of Ethyl Perfluorobutyl Ether and Ethyl Perfluoroisobutyl Ether is available as Cosmofluor76 from AE Chemie.

TABLE 2

| | range, w/w | range, w/w |
|---|---|---|
| PHASE A | | |
| Water (Aqua) | 10-40 | 25-35 |
| Xanthan Gum | 0-1 | 0.01-0.25 |
| Acrylates Copolymer | 0-20 | 0.1-15 |
| Glycereth-18 Ethylhexanoate | 0-10 | 0.1-5 |
| Glycereth-18 | | |
| Water | 1-50 | 25-45 |
| Sodium C14-16 Olefin Sulfonate | | |
| Sodium Cocoamphoacetate | | |
| Cocamidopropyl Hydroxysultaine | | |
| Propanediol | | |
| Phenoxyethanol | | |
| Ethylhexylglycerin | | |
| Sodium Chloride | | |
| Iron Oxide (CI 77491) | 0-1 | 0-1 |
| Disodium Carboxyethyl Siliconate | | |
| Titanium Dioxide (CI 77891) Disodium | 0-5 | 0-5 |
| Carboxyethyl Siliconate | | |
| Disodium EDTA | 0-1 | 0-1 |
| Coco Glucoside | 0-20 | 0.1-10 |
| Coconut Alcohol | | |
| Behenyl Alcohol | 0-5 | 0.1-2.5 |
| Stearyl Alcohol | 0-10 | 0.1-5 |
| colloidal sulfur | 0-15 | 3-10 |
| Ethylhexylglycerin | 0-5 | 0.1-2.5 |
| Phenoxyethanol | | |
| PHASE B | | |
| Sodium Hydroxide | 0-10 | 0.1-2.5 |
| Water (Aqua) | | |
| PHASE C | | |
| Avena Sativa (Oat) Extract | 0-5 | 0.1-2.5 |
| Water | | |
| Glycerin | | |
| Potassium sorbate | | |
| Water (Aqua) | 0-10 | 1-8 |
| Butylene Glycol | | |
| Oryza Sativa (Rice) Bran Extract | | |
| Boswellia Serrata Extract | | |
| Honey Extract | | |
| Oligopeplide-10 | | |
| Phenoxyethanol | | |
| Sodium Benzoate | | |
| Fragrance | 0-5 | 0.1-2.5 |
| PHASE D | | |
| Citric Acid | 0-5 | 0.1-2.5 |
| Water (Aqua) | | |
| PHASE E | | |
| Ethyl Perfluorobutyl Ether | 0-10 | 0.1-5 |
| Ethyl Perfluoroisobutyl Ether | | |

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above, are hereby incorporated in their entirety by reference.

What is claimed is:

1. A self-foaming composition comprising:
at least one perfluoro compound;
at least one surfactant; and
at least one fatty component selected from a fatty alcohol and/or a fatty acid;
wherein the composition, when released from a sealed vessel, forms a foam.

2. The composition of claim 1, comprising from about 0.1% w/w to about 25% w/w of surfactant(s).

3. The composition of claim 2, wherein the surfactant(s) are selected from glycereth-18 ethylhexanoate, glycereth-18, sodium C14-16 olefin sulfonate, sodium cocoamphoacetate, cocamidopropyl hydroxysultaine, coco glucoside, and mixtures thereof.

4. The composition of claim 2, comprising from about 0.1% w/w to about 10% w/w of the fatty component.

5. The composition of claim 4, wherein the fatty component is selected from cetyl alcohol, coconut alcohol, oleyl alcohol, stearyl alcohol, arachidyl alcohol, batyl alcohol, behenyl alcohol, lauric acid, myristic acid, palmitic acid, stearic acid, arachidic acid, behenic acid, myristoleic acid, palmitoleic acid, oleic acid, linoleic acid, linolenic acid, arachadonic acid, and combinations thereof.

6. The composition of claim 5, comprising at least two fatty components.

7. The composition of claim 1, further comprising a skin care active agent.

8. The composition of claim 7, wherein the skin care active agent is selected from sulfur, colloidal sulfur, benzoyl peroxide, and salicylic acid, or a combination thereof.

9. The composition of claim 1, wherein the composition comprises:
water (40-60% w/w);
at least one of: glycereth-18 ethylhexanoate (0-2.5% w/w), glycereth-18 (0-2.5% w/w), sodium C14-16 olefin sulfonate (5-15% w/w), sodium cocoamphoacetate (1-5% w/w), cocamidopropyl hydroxysultaine (0.1-1.5% w/w), and coco glucoside (2-8% w/w); and
at least one of: coconut alcohol (2-8% w/w), behenyl alcohol (0.1-2.5% w/w), and stearyl alcohol (0.1-5% w/w).

10. The composition of claim 1, wherein the composition comprises Water (Aqua), Xanthan Gum, Acrylates Copolymer, Glycereth-18 Ethylhexanoate, Glycereth-18, Sodium C14-16 Olefin Sulfonate, Sodium Cocoamphoacetate, Cocamidopropyl Hydroxysultaine, Propanediol, Phenoxyethanol, Ethylhexylglycerin, Coco Glucoside, Coconut Alcohol, Behenyl Alcohol, Stearyl Alcohol, Ethylhexylglycerin, Phenoxyethanol, and colloidal sulfur.

11. The composition of claim 1, wherein the perfluoro compound is a perfluoro ether.

12. The composition of claim 1 in a sealed bag-on-valve container.

13. A method of making a self-foaming composition, comprising:
forming a composition by combining at least one perfluoro compound; at least one surfactant; and at least one fatty component selected from a fatty alcohol and/or a fatty acid; and
enclosing the composition in a sealed vessel;
wherein the composition, when released from the sealed vessel, forms a foam.

14. The method of claim 13, wherein the composition comprises from about 0.1% w/w to about 25% w/w of surfactant(s).

15. The method of claim 13, wherein the surfactant(s) are selected from glycereth-18 ethylhexanoate, glycereth-18, sodium C14-16 olefin sulfonate, sodium cocoamphoacetate, cocamidopropyl hydroxysultaine, coco glucoside, and mixtures thereof.

16. The method of claim 13, wherein the composition comprises from about 0.1% w/w to about 10% w/w of the fatty component.

17. The method of claim 16, wherein the fatty component is selected from cetyl alcohol, coconut alcohol, oleyl alcohol, stearyl alcohol, arachidyl alcohol, batyl alcohol, and behenyl alcohol, lauric acid, myristic acid, palmitic acid, stearic acid, arachidic acid, behenic acid, myristoleic acid, palmitoleic acid, oleic acid, linoleic acid, linolenic acid, arachadonic acid, and combinations thereof.

18. The method of claim 17, comprising at least two fatty components.

19. The method of claim 13, wherein the composition further comprises a skin care active agent.

20. The method of claim 19, wherein the a skin care active agent is selected from sulfur, colloidal sulfur, benzoyl peroxide, and salicylic acid, or a combination thereof.

21. The method of claim 13, wherein the composition comprises:
water (40-60% w/w);
at least one of: glycereth-18 ethylhexanoate (0-2.5% w/w), glycereth-18 (0-2.5% w/w), sodium C14-16 olefin sulfonate (5-15% w/w), sodium cocoamphoacetate (1-5% w/w), cocamidopropyl hydroxysultaine (0.1-1.5% w/w), and coco glucoside (2-8% w/w); and
at least one of: coconut alcohol (2-8% w/w), behenyl alcohol (0.1-2.5% w/w), and stearyl alcohol (0.1-5% w/w)

22. The method of claim 13, wherein the composition comprises Water (Aqua), Xanthan Gum, Acrylates Copolymer, Glycereth-18 Ethylhexanoate, Glycereth-18, Sodium C14-16 Olefin Sulfonate, Sodium Cocoamphoacetate, Cocamidopropyl Hydroxysultaine, Propanediol, Phenoxyethanol, Ethylhexylglycerin, Coco Glucoside, Coconut Alcohol, Behenyl Alcohol, Stearyl Alcohol, Ethylhexylglycerin, Phenoxyethanol and colloidal sulfur.
